(19) United States
(12) Patent Application Publication
(10) Pub. No.: US 2025/0170164 A1
(43) Pub. Date: May 29, 2025

(54) PREPARATION PROCESS OF GLYCYRRHIZA POLYSACCHARIDE CAPABLE OF PROMOTING T CELL PROLIFERATION

(71) Applicant: KANGQI (TIANJIN) BIOTECHNOLOGY CO., LTD., Tianji (CN)

(72) Inventors: Fei SONG, Tianjin (CN); Xuefeng XIAO, Tianjin (CN)

(21) Appl. No.: 18/940,027

(22) Filed: Nov. 7, 2024

(30) Foreign Application Priority Data
Nov. 28, 2023 (CN) 202311595539.1
Mar. 14, 2024 (SG) 10202400710V

(51) Int. Cl. A61K 31/715; A61K 36/484

(52) U.S. Cl. CPC A61K 31/715; A61K 36/484; A61K 2236/53

(57) ABSTRACT

The present invention discloses a preparation process of glycyrrhiza polysaccharide capable of promoting T cell proliferation. The glycyrrhiza polysaccharide capable of promoting T cell proliferation can be obtained by extraction of glycyrrhiza polysaccharide, purification with activated carbon and modified adsorption resin, and then further removal of impurities by using DEAE-52 cellulose column for anion exchange, a membrane comprised of hollow fibers and Sephadex G-100 for adsorption. Compared with the prior art, the preparation process of glycyrrhiza polysaccharide of the present invention has the advantages that impurities such as pigments, proteins and the like in the glycyrrhiza polysaccharide can be removed, the content and the activity of glycyrrhiza polysaccharide are retained, the purity of the glycyrrhiza polysaccharide is improved, and the obtained glycyrrhiza polysaccharide has the effect of promoting T cell proliferation.

### TECHNICAL FIELD

The present invention is related to a technical field of medicine. It concerns more particularly a preparation process of glycyrrhiza polysaccharide capable of promoting T cell proliferation.

### BACKGROUND

Some preparation processes of currently known glycyrrhiza polysaccharide capable of promoting T cell proliferation include: an extraction method with ultrasonic assistance, using ultrasonic technology to assist extraction of glycyrrhiza polysaccharide, which can improve extraction efficiency and speed, due to the ultrasonic wave can destroy the cell structure so as to promote release of glycyrrhiza polysaccharide; an extraction method of enzymatic hydrolysis adopting special enzyme (such as cellulase, protease and so on) to carry out enzymolysis to glycyrrhiza, so as to release glycyrrhiza polysaccharide from the cell wall, which can improve the extraction rate and the purity of glycyrrhiza polysaccharide; an ion exchange method for purifying glycyrrhiza polysaccharide by ion exchange resin, which can selectively absorb and elute the target product according to the affinity between glycyrrhiza polysaccharide and the resin, so as to realize purification; a method of gel filtration chromatography, using gel filtration chromatography technology (such as Sephadex G-100 and so on) to separate and purify glycyrrhiza polysaccharide, which can separate glycyrrhiza polysaccharide according to its molecular size and shape so as to obtain the target product; and a method of ultra-filtration, using an interception film with suitable molecular weight to remove the impurity of the smaller molecules, so as to improve the purity of glycyrrhiza polysaccharide.

The ion exchange method is a common separation and purification technology and widely applied to the fields of biological pharmacy, food industry and environment analysis. It realizes the selective separation and purification of the target substance based on the adsorption and elution effect of the ion exchange resin to the ion or molecule in the substance. The ion exchange resin is a porous polymeric material with fixed ion groups. It can interact with charged ions or ionic molecules in an aqueous solution. The working process of the method of ion exchange is as follows: the mixed solution to be processed is passed through the column or bed layer filled with ion exchange resin, and the target ion or molecule can be adsorbed by the ion group on the ion exchange resin. Eluting, by changing the property of the solution, for example by adjusting the pH of the solution, the ionic strength or using a specific eluent, is able to disrupt the adsorption of the target substance by the ion exchange resin, so as to elute it from the resin. The eluted target substance is the purified product. It has advantages of strong selectivity, wide application, and high controllability.

However, in applications of the ion exchange method, factors such as the selection of the ion exchange resin, the optimization of the conditions for operation and the subsequent treatment of the eluted product need to be considered, so as to achieve desire purification effects.

Chinese patent CN109988795B discloses a process for separating and purifying glycyrrhiza polysaccharide, comprising the following steps: (1) cutting glycyrrhiza into slices; (2) soaking glycyrrhiza slices into water; (3) discarding the soaking solution, again adding water which is of 2 to 4 times of the weight of glycyrrhiza slices, repeatedly extracting glycyrrhiza slices for 2 to 4 times, and concentrating them under vacuum; (4) adding ethanol of volume fraction 92 to 97% to them, standing for 4 to 12 h, centrifuging, filtering, and drying them to obtain primary extract; adding ethanol of volume fraction 92 to 97% to the primary extract to soak for 2-6 h, centrifuging, filtering, drying the primary extract, and repeating for 2-4 times to obtain glycyrrhiza polysaccharide. The process for separating and purifying of glycyrrhiza polysaccharide of the present invention facilitates the leaching of glycyrrhiza polysaccharide by freezing glycyrrhiza and enzymolysis under ultrasonic assistances to destroy the cell wall of glycyrrhiza, then undergoing the fermentation process, and subsequent separation and purification. The extraction process is simple and easy to carry out, and the yield and content of glycyrrhiza polysaccharide is high. The obtained glycyrrhiza polysaccharide has high activities, a low residual rate of the protein, high purity, and the function of regulating the body's immunity and eliminating fatigue. However, the process of separating and purifying glycyrrhiza polysaccharide of the invention has disadvantages in the removal rate of the protein and the retention rate of polysaccharide.

### SUMMARY

In view of the disadvantages of low removal rate of the protein and low retention rate of the polysaccharide in the process of separating and purifying glycyrrhiza polysaccharide in prior arts, the technical problem to be solved by the present invention is to provide a preparation process of glycyrrhiza polysaccharide capable of promoting T cell proliferation with high removal rate of the protein and the retention rate of polysaccharide.

In order to achieve the above-mentioned object of the present invention, the present invention provides following technical solutions:

A preparation technique of glycyrrhiza polysaccharide capable of promoting T cell proliferation is as follows, comprises:

Z1, weighing 4 to 6 parts by weight of precipitate of glycyrrhiza polysaccharide, adding water to it to prepare solution of glycyrrhiza polysaccharide, heating and boiling for 5 to 15 min to dissolve the precipitate, adding 1 to 3 wt % of activated carbon, continuously boiling the solution for 5 to 15 min to absorb pigment, centrifuging firstly and collecting supernatant, then centrifuging secondly and collecting supernatant, both of the supernatant of which is purified through modified adsorption resin by column chromatography purification, collecting the effluent, filtering the effluent by using filter cloth of 100-300 meshes to obtain the decoloured and deproteinized glycyrrhiza polysaccharide solution;
Z2, adding the glycyrrhiza polysaccharide solution prepared in the step Z1 into the DEAE-52 cellulose column bed for anion exchange, eluting the glycyrrhiza polysaccharide with ultrapure water, collecting the eluent, drawing the eluting curve of the DEAE-52 cellulose for anion exchange to collect the eluting peak, obtaining eluting solution of the glycyrrhiza polysaccharide; removing the impurities of small molecules in the eluting solution of the glycyrrhiza polysaccharide by using a membrane comprised of hollow fibers to obtain the glycyrrhiza polysaccharide solution with the impurities of small molecules removed; evaporating and concentrating the glycyrrhiza polysaccharide solution with the impurities of small molecules removed to 1 to 3 mg/mL, adding it to the Sephadex G-100 column bed of glucan gel, eluting it by ultrapure water, collecting the eluent while drawing the elution curve of the Sephadex G-100 column of glucan gel to collect the absorption peak, freezing and drying the eluent to obtain the glycyrrhiza polysaccharide capable of promoting T cell proliferation.

Preferably, in the step Z1, adding water to prepare solution of glycyrrhiza polysaccharide of 8-12 mg/mL.

Preferably, in the step Z1, centrifuging firstly for 10-30 min at 3000-5000 rpm and collecting the supernatant, and centrifuging secondly for 10-20 min at 10000-12000 rpm.

Preferably, in the step Z1, parameters of the column chromatography purification are as follows: diameter-to-height ratio is 1:5-20, flow speed is 1-3 BV/h, and time for dynamic adsorption is 10-30 min.

The preparation method of the precipitate of the glycyrrhiza polysaccharide comprises:
step 1, weighing 8-12 parts by weight of glycyrrhiza powder, adding water to it, ultrasonically processing at 30-40° C., filtering it and collecting the filtrate and the filter residue;
step 2, adding water to the filter residue in the step 1, refluxing the solution for 1-3 h in the boiling water bath, then filtering the solution, collecting the filtrate and combining it with the filtrate obtained in the step 1, and then centrifuging for separation and collecting the supernatant;
step 3, concentrating the supernatant prepared in the step 2 to ⅕ to 1/10, then adding anhydrous ethanol of 3 to 5 times volume of the concentrated supernatant, sealing the solution, placing it at −1 to −10 C°, standing for 20 to 30 h to separate out glycyrrhiza polysaccharide precipitate.

Preferably, water is added in said step 1 and step 2 according to mass ratio of 1:20-30.

Preferably, in step 1, time for the ultrasonic process is 40-60 min, power of ultrasonic wave thereof is 200-400 W and frequency of the ultrasonic wave thereof is 20-60 kHz.

The preparation method of the modified adsorption resin comprises:
stirring and mixing the amino resin and the glyoxal, and then adding the diethyl cyanophosphate, heating it at 80-120° C. for 20-40 min to obtain chelating resin; adding the copper naphthenate, water and the macroporous absorption resin, placing it onto a vibration table, shaking and vibrating it at 25 to 40° C., filtering it, collecting the solid and washing it with water, to obtain the modified absorption resin.

Preferably, the preparation method of the modified adsorption resin comprises:
stirring and mixing 18-22 parts by weight of the amino resin and 8-12 parts by weight of glyoxal for 1-3 h at 100-300 rpm, and then adding 2-4 parts by weight of the diethyl cyanophosphate, heating it at 80-120° C. for 20-40 min to obtain chelating resin; adding 10 to 20 parts by weight of copper naphthenate, 500 to 700 parts by weight of water and 30 to 50 parts by weight of the macroporous absorption resin, placing it onto a vibration table, shaking and vibrating it for 0.5 to 2 h at 200 to 300 rpm at 25 to 40° C., filtering it with a filter cloth of 50 to 300 mesh, collecting the solid and washing it with water to obtain the modified absorption resin.

Glycyrrhiza polysaccharide is a polysaccharide compound extracted from roots of glycyrrhizas. Glycyrrhizas are a traditional herbal medicine which is widely used in traditional Chinese medicine and have play an important role in food and medicine in many countries.

The glycyrrhiza polysaccharide has multiple biological activities and pharmacological actions, including immunoregulation, anti-inflammatory action, anti-oxidation action and anti-tumour action. In addition, the glycyrrhiza polysaccharide also has multiple pharmacological effects, such as resisting ulcer, resisting virus, resisting allergy, reducing blood sugar and so on.

The present invention can effectively absorb and remove the proteins in the solution of the glycyrrhiza polysaccharide by adding the modified absorbing resin, which has specific surface properties and affinity, and can selectively absorb the protein molecules so as to improve the removal rate of the proteins. This facilities purification the solution of the glycyrrhiza polysaccharide, removing the protein impurities therein and hence improving purity of the glycyrrhiza polysaccharide. The modified adsorbent resin also facilitates good retention properties of the polysaccharide. In the process of preparing the glycyrrhiza polysaccharide solution, the modified absorption resin can selectively absorb other impurities of small molecules, such as pigment and compounds with low molecular weight, while providing low absorption ability to the glycyrrhiza polysaccharide. As a result, the retention rate of the glycyrrhiza polysaccharide in the purification process can be increased so as to avoid loss of the glycyrrhiza polysaccharide, and the activities and functionalities of the glycyrrhiza polysaccharide can be retained.

The modified adsorption resin introduces functional groups with specific functions (such as amino, phosphate and so on) through regulation of surface properties to regulate affinity and charge properties of the surface of the resin. Adsorption happens between these functional groups and the target molecules (such as protein) through forces caused by electrostatic interaction, hydrogen bond and Van der Waals force and so on, so that selective adsorption and removal can be realized. The modified adsorption resin has a certain of pore sizes and pore structures, and can selectively adsorb molecules through effect of size exclusion. Larger molecules (such as polysaccharides) are often unable to enter the pores of the resin due to their larger molecular sizes, and thus are effectively retained, while the smaller molecules (such as protein) can enter the pores and be absorbed, resulting in realization of separation and purification. Specific affinity can happen between the functional groups in the modified adsorption resin and the target molecules. For example, there are groups in some modified adsorbent resins which have affinity with ligands in chromatography columns, and can selectively bind to specific domains or functional groups of proteins. High efficiency of adsorption and separation of specific protein can be realized by this affinity. To summarize, through introduction of the modified adsorption resin, the surface properties and the pore structures can be regulated to realize the selective adsorption and separation of the protein and the polysaccharide, and hence improve the removal rate of the protein and the retention rate of the polysaccharide. These results are mainly achieved by mechanisms such as regulation of surface properties, effect of size exclusion and effect of affinity adsorption. Furthermore, structures of the polysaccharide will not be destroyed by processes of deproteinization and decolourization with the method of the modified adsorbent resin.

Compared with the prior arts, many beneficial advantages can be achieved by the present invention.

In the present invention, by using activated carbon and modified adsorption resin to remove impurities such as pigments and proteins in glycyrrhiza polysaccharide, contents and activities of the glycyrrhiza polysaccharide are retained, and purity of the glycyrrhiza polysaccharide is improved.

In the present invention, by using the cellulose columns of DEAE-52 for anion exchange and the membrane comprised of hollow fibers, the impurities of small molecules in the solution of the glycyrrhiza polysaccharide solution can be removed to further improve the purity of the glycyrrhiza polysaccharide.

In the present invention, by using of glucan gel columns of Sephadex G-100 to realize fine separation of the glycyrrhiza polysaccharide and collection of the absorption peak according to elution curve, the target glycyrrhiza polysaccharide can be obtained.

In the present invention, the glycyrrhiza polysaccharide obtained by the above-mentioned steps of purification can provide the effect of promoting T cell proliferation, and can provide certain effects of immunoregulations and immunoenhancements.

### BRIEF DESCRIPTION OF THE FIGURES

FIG. 1 is a standard curve of protein in a test example 1.

FIG. 2 is a standard curve of glucose in a test example 2.

### DETAILED DESCRIPTION

### Main Material Sources:

Amino resin: Guangzhou Lingdi New Material Co., Ltd., Type No.: CYMEL 3031 F.

Macroporous absorption resin: Tianjin Bohong Resin Technology Co., Ltd., Item No.: BH4006.

Glycyrrhiza powder: Xian Entaiyuan Biological Technology Co., Ltd., Item No.: ety114.

Activated carbon: Shandong Musen Carbon Co., Ltd., Particle size: 200-325 screen mesh, Item No.: powder 4.

### Example 1

A preparation process of glycyrrhiza polysaccharide capable of promoting T cell proliferation comprises:

Z1, weighing 5 g of precipitate of glycyrrhiza polysaccharide, adding water to it to prepare glycyrrhiza polysaccharide solution of 10 mg/mL, heating and boiling the solution for 10 min to dissolve the precipitate of glycyrrhiza polysaccharide, then adding 2 wt % of activated carbon, continuously boiling the solution for 10 min to absorb pigment, centrifuging firstly for 20 min at 4000 rpm and collecting supernatant, then centrifuging secondly for 15 min at 11000 rpm and collecting the supernatant again, both of the supernatant of which is purified through modified adsorption resin by column chromatography purification with the column diameter-to-height ratio 1:10 of the resin, the flow speed 2 BV/h, and the dynamic adsorption time for 20 min, collecting the effluent, and filtering the effluent by using filter cloth of 200 meshes to obtain the decoloured and deproteinized glycyrrhiza polysaccharide solution; and

Z2, adding the glycyrrhiza polysaccharide solution prepared in the step Z1 into the DEAE-52 cellulose column bed for anion exchange, eluting the glycyrrhiza polysaccharide with ultrapure water, collecting the eluent to obtain eluting solution of the glycyrrhiza polysaccharide while drawing the eluting curve of the DEAE-52 cellulose for anion exchange to collect the eluting peak; removing the impurities of small molecules in the eluting solution of the glycyrrhiza polysaccharide by using a membrane comprised of hollow fibers to obtain the glycyrrhiza polysaccharide solution with the impurities of small molecules removed; evaporating and concentrating the glycyrrhiza polysaccharide solution with the impurities of small molecules removed to 2 mg/mL, adding it to the Sephadex G-100 column bed of glucan gel, eluting it by ultrapure water, collecting the eluent while drawing the elution curve of the Sephadex G-100 column of glucan gel to collect the absorption peak, freezing and drying the eluent to obtain the glycyrrhiza polysaccharide capable of promoting T cell proliferation.

The preparation method of the precipitate of glycyrrhiza polysaccharide comprises:
step 1, weighing 10 g of glycyrrhiza powder, adding water to it according to the mass ratio of 1:25, undergoing ultrasonic process for 50 min at 35° C., with the power of ultrasonic wave 300 W and the ultrasonic frequency 40 kHz, filtering it and collecting the filtrate and the filter residue;
step 2, adding water to the filter residue in the step 1 according to the mass ratio of 1:25 to prepare solution, refluxing the solution for 1.5 h in the boiling water bath, then filtering the solution, collecting the filtrate and combining it with the filtrate obtained in the step 1, and then centrifuging for separation and collecting the supernatant;
step 3, concentrating the supernatant prepared in the step 2 to ⅛, adding anhydrous ethanol of 4 times volume of the concentrated supernatant to prepare a solution, sealing the solution and then standing it at −4° C. for 24 h to separate out the precipitate of the glycyrrhiza polysaccharide.

The preparation method of the modified adsorption resin comprises:

Stirring and mixing 20 g of the amino resin and 10 g of the glyoxal for 2 h at 200 rpm to prepare a mixture, and then adding 3 g of the diethyl cyanophosphate to the mixture to prepare a solution and heating it at 100° C. for 30 min to obtain a chelate resin; adding 15 g of copper naphthenate, 600 g of water, and 40 g of the macroporous absorption resin to the chelate resin to prepare a solution and placing it onto a vibration table, shaking and vibrating it at 240 rpm at 30° C. for 1 hour, filtering it with a filter cloth of 100 mesh, collecting the solid and washing it with water to obtain the modified absorption resin.

### Comparative Example 1

A preparation process of glycyrrhiza polysaccharide capable of promoting T cell proliferation is substantially the same as example 1, and the difference only lies in that the preparation method of the modified adsorption resin is different.

The preparation method of the modified adsorption resin comprises:

Stirring and mixing 20 g of the amino resin and 10 g of the acetaldehyde for 2 h at 200 rpm to prepare a mixture, and then adding 3 g of the diethyl cyanophosphate to the mixture to prepare a solution and heating it at 100° C. for 30 min to obtain a chelate resin; adding 15 g of copper naphthenate, 600 g of water, and 40 g of the macroporous absorption resin to the chelate resin to prepare a solution and placing it onto a vibration table, shaking and vibrating it at 240 rpm at 30° C. for 1 hour, filtering it with a filter cloth of 100 mesh, collecting the solid and washing it with water to obtain the modified absorption resin.

### Comparative Example 2

A preparation process of glycyrrhiza polysaccharide capable of promoting T cell proliferation is substantially the same as example 1, and the difference only lies in that the preparation method of the modified adsorption resin is different.

The preparation method of the modified adsorption resin comprises:

Stirring and mixing 20 g of the amino resin and 10 g of the glyoxal for 2 h at 200 rpm to prepare a mixture, and then adding 3 g of the Ethylene glycol phosphate to the mixture to prepare a solution and heating it at 100° C. for 30 min to obtain a chelate resin; adding 15 g of copper naphthenate, 600 g of water, and 40 g of the macroporous absorption resin to the chelate resin to prepare a solution and placing it onto a vibration table, shaking and vibrating it at 240 rpm at 30° C. for 1 hour, filtering it with a filter cloth of 100 mesh, collecting the solid and washing it with water to obtain the modified absorption resin.

### Comparative Example 3

A preparation process of glycyrrhiza polysaccharide capable of promoting T cell proliferation is substantially the same as example 1, and the difference only lies in that the preparation method of the modified adsorption resin is different.

The preparation method of the modified adsorption resin comprises:

Stirring and mixing 20 g of the amino resin and 10 g of the acetaldehyde for 2 h at 200 rpm to prepare a mixture, and then adding 3 g of the diethyl cyanophosphate to the mixture to prepare a solution and heating it at 100° C. for 30 min to obtain a chelate resin; adding 15 g of zinc sulfate, 600 g of water, and 40 g of the macroporous absorption resin to the chelate resin to prepare a solution and placing it onto a vibration table, shaking and vibrating it at 240 rpm at 30° C. for 1 hour, filtering it with a filter cloth of 100 mesh, collecting the solid and washing it with water to obtain the modified absorption resin.

### Comparative Example 4

A preparation process of glycyrrhiza polysaccharide capable of promoting T cell proliferation is substantially the same as example 1, and the difference only lies in that the preparation method of the modified adsorption resin is different.

The preparation method of the modified adsorption resin comprises:

Stirring and mixing 20 g of the amino resin and 10 g of the acetaldehyde for 2 h at 200 rpm to prepare a mixture, and then adding 3 g of the Ethylene glycol phosphate to the mixture to prepare a solution and heating it at 100° C. for 30 min to obtain a chelate resin; adding 15 g of zinc sulfate, 600 g of water, and 40 g of the macroporous absorption resin to the chelate resin to prepare a solution and placing it onto a vibration table, shaking and vibrating it at 240 rpm at 30° C. for 1 hour, filtering it with a filter cloth of 100 mesh, collecting the solid and washing it with water to obtain the modified absorption resin.

### Test Example 1

Test of removal rate of the Protein

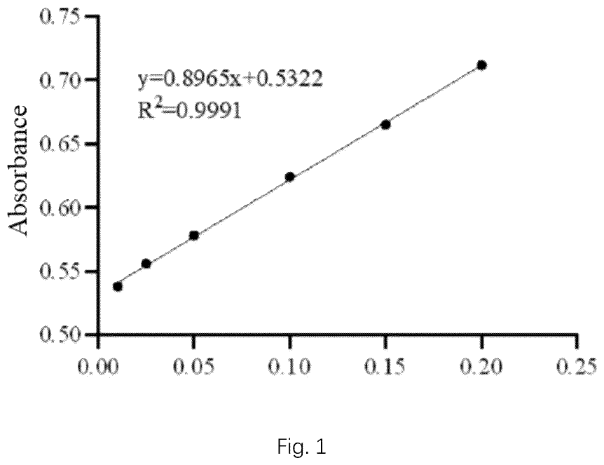

Precisely weighing 10.00 mg of bovine serum albumin (BSA), placing it in a 10 mL volumetric flask to prepare a standard solution of 1 mg/mL bovine serum albumin (BSA). Diluting the standard solution in gradient to 0.01 mg/mL, 0.025 mg/mL, 0.05 mg/mL, 0.1 mg/mL, 0.15 mg/mL, and 0.2 mg/mL. Preparing dyeing solution of 1×G250 Coomassie brilliant blue by taking 1 mL of dyeing solution of 5×Coomassie brilliant blue and add 4 mL of ultrapure water to it. Transferring 20 μL of above-mention a serial of standard solutions of the protein respectively into a 96-hole plate, adding 200 μL of 1×Coomassie brilliant blue dye solution into each of the standard solutions, standing for 3 min for reaction, and then using an enzyme labelling instrument to measure the absorbance at 595 nm, while drawing the standard curve of the protein, as shown in FIG. 1, with the concentration of the protein as a horizontal coordinate, and the absorbance as a longitudinal coordinate.

The regression equation of the concentration of the protein and the absorbance in the range of 0.01 mg/mL˜0.20 mg/mL is: Y=0.8965x+0.5322, and the correlation coefficient R²=0.9991. Prepare the solutions of glycyrrhiza polysaccharide of 10 mg/mL for protein test by taking the glycyrrhiza polysaccharide of the invention prepared before and after purification and adding water to it.

Y₁(%)=(N₀−N₁)/N₀×100
wherein: Y1 is the protein removing rate with unit of %;
N₀ is the content of the protein before purification with unit of mg;
N₁ is the content of the protein after purification with unit of mg.

Each group was tested three times, the average values were calculated, and the test results were shown in Table 1.

TABLE 1
Test results of the removal rate of the protein

| | Removal rate |
|---|---|
| Experiment solution | of protein/% |
| Example 1 | 76.27 |
| Comparative example 1 | 59.96 |
| Comparative example 2 | 54.71 |
| Comparative example 3 | 51.02 |
| Comparative example 4 | 31.75 |

### Test Example 2

### Test of Retention Rate of Polysaccharide

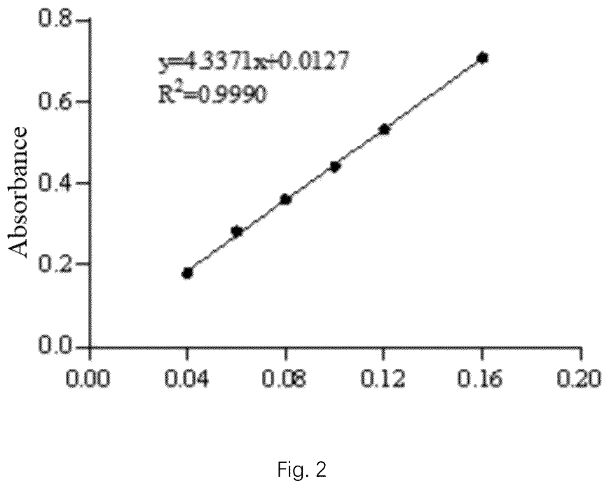

The content of the polysaccharide is measured by phenol-sulphuric acid method: precisely weighing 100.00 mg of anhydrous glucose which has been dried with constant weight, placing it in a volumetric flask dissolving it with ultrapure water and adding the ultrapure water to a constant volume of 100 mL to prepare into standard solution of glucose of 1 mg/mL, diluting it in gradient to 0.04, 0.06, 0.08, 0.10, 0.12 0.16 mg/mL, taking 1 mL of each of solutions in gradient concentration in a brown volumetric flask of 10 ml respectively, adding 1 mL of 5% phenol solution to each of flasks, shaking them up, quickly adding 5 mL of concentrated sulfuric acid to each of flasks, shaking them up and standing them at room temperature for 30 min, adding ultrapure water to each of flasks to a constant volume of 10 ml, cooling them to room temperature via bath of ice water, and measuring the absorbance of each of flasks using an ultraviolet spectrophotometer at 490 nm, while drawing the standard curve of the glucose, as shown in FIG. 2, with the concentration of the glucose as a horizontal coordinate, and the absorbance as a longitudinal coordinate.

The regression equation of the concentration of the glucose and the absorbance in the range of 0.04 mg/mL-0.16 mg/mL is y=4.3371x+0.0127, and the correlation coefficient R²=0.9990. The results show that the established measuring method for the content of the glycyrrhiza polysaccharide presents a good linear relationship in the stated range of the concentration.

The content of polysaccharide is measured by phenol-sulphuric acid method: using a liquid transferring gun to absorb 10 mg of each of solutions of the glycyrrhiza polysaccharide prepared according to the present invention to a volumetric flask of 100 mL, adding 200 μL of 5% phenol solution to each of flasks, after shaking them up, quickly adding 1 mL of 98% concentrated sulphuric acid to each of flasks, shaking them up, standing them for 5 min, heating them for 30 min in the water bath of 37 C°, cooling them at room temperature, measuring the absorbance for each at 488 nm and calculating the yield of the glycyrrhiza polysaccharide according to the below formula.

Retention rate of polysaccharide=concentration of polysaccharide before purification/concentration of polysaccharide after purification*100%

Each group was tested three times, and the average value was calculated, and the test results were shown in Table 2.

TABLE 2
Test results of the retention rate of the polysaccharide

| | Retention rate of |
|---|---|
| Experiment solution | polysaccharide |
| Example 1 | 92.52 |
| Comparative example 1 | 77.61 |
| Comparative example 2 | 78.33 |
| Comparative example 3 | 76.42 |
| Comparative example 4 | 57.48 |

From the results of the test examples 1 and 2, it can be seen that the glycyrrhiza polysaccharide prepared in the embodiment 1 of the present invention has high removal rate of the protein and retention rate of the polysaccharide. Comparing example 1 with comparative Example 1, the possible reasons can be found in that there are more reaction sites in the present invention using the glyoxal, and thus more functional groups can be provided to react with amino groups on the resin to form more affinity bonds, which increases the affinity of the surface of the modified adsorption resin, improves the selective adsorption ability to the protein resulting in the improved removal rate of the protein, and increases the stability of the pores of the resin and the uniformity of the size distribution resulting in the improved separation effect of the modified adsorbent resin and the increased retention rate of the polysaccharide in the purification process.

Comparing example 1 with comparative example 2, the possible reasons can be found in that in the present invention, the diethyl cyanophosphate is used in the preparation of the modified adsorption resin, and the functional groups introduced by the cyan of it have a stronger affinity than those introduced by the diethyl phosphate, which can form bonds to the protein molecules with more stable affinity, and adjust the affinity and charge properties of the surface of the resin surface. Adsorption happens between these functional groups and the target molecules (such as protein) through forces caused by electrostatic interaction, hydrogen bond and Van der Waals force and so on, so that selective adsorption and removal can be realized.

Comparing example 1 with comparative example 3, the possible reasons can be found in that the copper naphthenate is used in the preparation of modified adsorbent resin, which has a stronger selective adsorption ability to proteins. This means that it is easier for the adsorbent resin to adsorb protein molecules due to it can form a stronger interaction with the protein molecules, hence adsorb less polysaccharide molecules. In contrast, zinc sulfate may have weaker selective adsorption ability to proteins and polysaccharides during the adsorption process.

1. A preparation process of glycyrrhiza polysaccharide capable of promoting T cell proliferation, comprises
Z1, weighing 4 to 6 parts by weight of precipitate of glycyrrhiza polysaccharide, adding water to it to prepare solution of glycyrrhiza polysaccharide, heating and boiling for 5 to 15 min to dissolve the precipitate, adding 1 to 3 wt % of activated carbon, continuously boiling the solution for 5 to 15 min to absorb pigment, centrifuging firstly and collecting supernatant, then centrifuging secondly and collecting supernatant, both of the supernatant of which is purified through modified adsorption resin by column chromatography purification, collecting the effluent, filtering the effluent by using filter cloth of 100-300 meshes to obtain the decoloured and deproteinized glycyrrhiza polysaccharide solution;
Z2, adding the glycyrrhiza polysaccharide solution prepared in the step Z1 into the DEAE-52 cellulose column bed for anion exchange, eluting the glycyrrhiza polysaccharide with ultrapure water, collecting the eluent, drawing the eluting curve of the DEAE-52 cellulose for anion exchange to collect the eluting peak, obtaining eluting solution of the glycyrrhiza polysaccharide; removing the impurities of small molecules in the eluting solution of the glycyrrhiza polysaccharide by using a membrane comprised of hollow fibers to obtain the glycyrrhiza polysaccharide solution with the impurities of small molecules removed; evaporating and concentrating the glycyrrhiza polysaccharide solution with the impurities of small molecules removed to 1 to 3 mg/mL, adding it to the Sephadex G-100 column bed of glucan gel, eluting it by ultrapure water, collecting the eluent while drawing the elution curve of the Sephadex G-100 column of glucan gel to collect the absorption peak, freezing and drying the eluent to obtain the glycyrrhiza polysaccharide capable of promoting T cell proliferation;
wherein the preparation method of the precipitate of the glycyrrhiza polysaccharide comprises:
step 1, weighing 8-12 parts by weight of glycyrrhiza powder, adding water to it, ultrasonically processing at 30-40° C., filtering it and collecting the filtrate and the filter residue;
step 2, adding water to the filter residue in the step 1, refluxing the solution for 1-3 h in the boiling water bath, then filtering the solution, collecting the filtrate and combining it with the filtrate obtained in the step 1, and then centrifuging for separation and collecting the supernatant;
step 3, concentrating the supernatant prepared in the step 2 to ⅕ to 1/10, then adding anhydrous ethanol of 3 to 5 times volume of the concentrated supernatant, sealing the solution, placing it at −1 to −10 C°, standing for 20 to 30 h to separate out glycyrrhiza polysaccharide precipitate;
wherein the preparation method of the modified adsorption resin comprises:
stirring and mixing 18-22 parts by weight of the amino resin and 8-12 parts by weight of glyoxal for 1-3 h at 100-300 rpm, and then adding 2-4 parts by weight of the diethyl cyanophosphate, heating it at 80-120° C. for 20-40 min to obtain chelating resin; adding 10 to 20 parts by weight of copper naphthenate, 500 to 700 parts by weight of water and 30 to 50 parts by weight of the macroporous absorption resin, placing it onto a vibration table, shaking and vibrating it for 0.5 to 2 h at 200 to 300 rpm at 25 to 40° C., filtering it with a filter cloth of 50 to 300 mesh, collecting the solid and washing it with water to obtain the modified absorption resin.

2. A preparation process of glycyrrhiza polysaccharide capable of promoting T cell proliferation according to claim 1, wherein in step Z1, adding water to prepare solution of glycyrrhiza polysaccharide of 8-12 mg/mL.

3. A preparation process of glycyrrhiza polysaccharide capable of promoting T cell proliferation according to claim 1, wherein in the step Z1, centrifuging firstly for 10-30 min at 3000-5000 rpm and collecting the supernatant, and centrifuging secondly for 10-20 min at 10000-12000 rpm.

4. A preparation process of glycyrrhiza polysaccharide capable of promoting T cell proliferation according to claim 1, wherein in the step Z1, parameters of the column chromatography purification are as follows: diameter-to-height ratio is 1:5-20, flow speed is 1-3 BV/h, and time for dynamic adsorption is 10-30 min.

5. A preparation process of glycyrrhiza polysaccharide capable of promoting T cell proliferation according to claim 1, wherein water is added in said step 1 and step 2 according to mass ratio of 1:20-30.

6. A preparation process of glycyrrhiza polysaccharide capable of promoting T cell proliferation according to claim 1, wherein in step 1, time for the ultrasonic process is 40-60 min, power of ultrasonic wave thereof is 200-400 W and frequency of the ultrasonic wave thereof is 20-60 kHz.
